(19) United States
(12) Patent Application Publication
(10) Pub. No.: US 2024/0197192 A1
(43) Pub. Date: Jun. 20, 2024

(54) SYSTEM, METHOD AND COMPUTER PROGRAM FOR MONITORING HEALTH OF A PERSON

(71) Applicant: MERU HEALTH OY, Helsinki (FI)

(72) Inventors: Kristian RANTA, Helsinki (FI); Albert NAZANDER, Helsinki (FI)

(21) Appl. No.: 18/554,484

(22) PCT Filed: Apr. 11, 2022
(86) PCT No.: PCT/FI2022/050234

§ 371 Date: Oct. 9, 2023

(30) Foreign Application Priority Data
Apr. 12, 2021 (FI) 20215425

(51) Int. Cl. A61B 5/024; A61B 5/00; G16H 40/67

(52) U.S. Cl. CPC A61B 5/02405; A61B 5/486; A61B 5/7289; G16H 40/67

(57) ABSTRACT

A system for monitoring health of a person is disclosed. The system includes measuring heart beats of the person. The system also includes data processing system, which is configured to calculate instantaneous heart rate data and provide a periodic biofeedback function including parameters, the parameters including an amplitude A, and an angular frequency P. The periodic biofeedback function also includes a cumulative difference E relative to the instantaneous heart rate data. The data processing system is configured to fit the periodic biofeedback function into the instantaneous heart rate data and determine the parameters and cumulative difference E. The amplitude A determines a heart rate variability of the person. A related method and a computer program are also disclosed.

### FIELD OF THE INVENTION

The present invention relates to a system for monitoring health of a person and particularly to a system according to preamble of claim 1. The present invention relates also to a method for monitoring health of a person and particularly to a method according to preamble of claim 18. The present invention relates also to a computer program for monitoring health of a person and particularly to a computer program according to preamble of claim 19.

### BACKGROUND OF THE INVENTION

Vagus nerve is an important nerve in the human body and it plays a major role in the correct functioning of the parasympathetic nervous system. In the medical community, stimulation of the vagus nerve is believed to lead to various cardiovascular, cerebrovascular, metabolic and other physiological and mental health benefits.

In the prior art, vagus nerve has been stimulated in a non-invasive way for example by application of electricity to the frontal neck area of the human body. Devices to this purpose are commercially available, and they resemble an electrical razor apparatus in size and shape. Also invasive ways of vagus nerve stimulation exist. As an example, a device may be surgically implanted under the skin of a person's chest, and a wire may be threaded under the skin to connect the device to the vagus nerve. When activated, the device sends electrical signals along the vagus nerve to person's brainstem, activating the vagus nerve and the nervous system in general.

It is also known that breathing in a certain, in general, slow breathing rate may stimulate the vagus nerve favourably. As one way to stimulate the vagus nerve, breathing with a certain slow breathing rate offers many advantages as it involves no invasive or non-invasive electrical stimulation of the nerve which can be difficult to arrange especially if the related arrangements require surgical procedures or dedicated electrical devises or both. However, breathing analysis with the prior art is challenging as monitoring breathing requires its own set of dedicated devices. These devices may be based, for example, to the analysis of flow of breathing inhalations and exhalations and to the movement of breathing gasses in the respiratory tract, for example mouth or nose. Clearly, wearing or holding such metering devices in mouth or by the nose is relatively unpleasant and complex. Thus, one of the problems in the prior art is also the determination of the true measured breathing rate, the inhalations and exhalations of a person actually takes at any given time

Most effective positive vagus nerve stimulus through breathing is achieved when breathing rate of a person follows the natural heart rate variation of the person. In the medical community, this stimulation is sometimes called “cross-coherence” between breathing and heart rate variation. In general, when a person inhales, the instantaneous heart rate increases, and when a person exhales, the instantaneous heart rate decreases.

Normally at rest, an average heart rate is between 60 and 100 BPM. (beats per minute). However, this is heavily dependent on the gender, age, fitness level and level of relaxation of the person. Heart rate variations and breathing are also personal traits and there is no one universal value for a correct breathing rate to achieve strong vagus nerve stimulus through cross-coherence. Instead, every person has his or her own best breathing rate that best matches the heart rate variability (“HRV”) and breathing. Usually, the breathing rate providing the strongest vagus nerve stimulus is between 3 and 8 breaths in one minute. In general, a high heart rate variability in the heart rate is an indication of a strong vagus nerve stimulation with positive health effects.

In the prior art, there are some notions of analysing the cross-coherence of breathing rate and heartbeat rate to optimally stimulate the vagus nerve through breathing by trying to synchronize the breathing events and the heart rate variability. This has involved the usage of Fourier transforms and complex signal processing techniques to indicate spectral characteristics of heart rate variation and breathing. To get reliable spectral data, a very long dataset comprising data of instantaneous heart rates and breathing spanning several minutes has been needed. This makes the practical implementation of the associated optimal vagus nerve stimulation very difficult as the person under analysis needs to remain in complete rest, and at the same time, pay attention to the way he/she breathes and, possibly, alter the way he/she breathes for several minutes.

Preferably, vagus nerve stimulation through breathing is attempted frequently, somewhat like a repeated fitness routine or exercise, as the positive health effects may grow gradually stronger. With repeated breathing exercises, a person may achieve the health benefits faster during the exercise. Optimal breathing rate for vagus nerve stimulation may also decrease somewhat over time. Clearly, a comfortable and fast approach for determining the heart rate variation, and a related indication of the strength of the related vagus nerve stimulation through breathing is needed.

Thus, at least two prior art problems can be raised: Stimulating the vagus nerve requires complex devices and procedures and there is a clear need to overcome the above-mentioned problems in the prior art. If vagus nerve is to be stimulated with a correct breathing rate, determining the breathing rate which is well suited for a person to stimulate the vagus nerve through breathing with prior art methods and devices has been challenging. Strong vagus nerve stimulation is often indicated with a high heart rate variability (HRV), but determining this figure of merit, HRV, has been difficult with prior art solutions. Further, the mere determination of the true, measured breathing rate of a person with prior art devices is challenging. This is especially true if the determination of the true breathing rate is to be done during a relaxed and comfortable health monitoring session with a goal of stimulating the vagus nerve, without complex measurement devices in or by the respiratory tract.

### BRIEF DESCRIPTION OF THE INVENTION

An object of the present invention is to provide a system, a method and a computer program for monitoring health of a person during a health monitoring session so that the prior art disadvantages are solved or at least alleviated. The objects of the invention are achieved by a system according to the independent claim 1. The objects of the invention are further achieved by a method according to the independent claim 18. The objects of the invention are further achieved by a computer program according to the independent claim 25.

The preferred embodiments of the invention are disclosed in the dependent claims.

The present invention is based on an idea of providing a system A system for monitoring health of a person during a health monitoring session. The system comprises a heart rate sensor arranged to measure heart beats of the person for providing heart rate measurement data, and a data processing system configured to:
A) receive the heart rate measurement data from the heart rate sensor during the health monitoring session, and
C) analyse the heart rate measurement data such that in the analysis, the data processing system is configured to:
   C1) calculate instantaneous heart rate data comprising heart rate of each measured heartbeat, calculating performed over a time window in the health monitoring session, the time window comprising a timepoint, the instantaneous heart rate data being calculated based on the heart rate measurement data received from the heart rate sensor,
   C2) provide a periodic biofeedback function comprising parameters and a cumulative difference E relative to the instantaneous heart rate data, the parameters comprising:
      an amplitude A, the amplitude A determining a heart rate variability of the person at the timepoint, and
      an angular frequency P of the periodic biofeedback function at the timepoint,
   C3) fit the periodic biofeedback function into the instantaneous heart rate data within the time window to determine
      the parameters of the periodic biofeedback function at the timepoint, and
      the cumulative difference E at the timepoint.

Advantage of the system is that the heart rate variability, among other relevant parameters of the periodic biofeedback function, are determined quickly when compared to prior art systems, without complex arrangements for example in the respiratory tract, or having to gather a large dataset to get a reliable Fourier transform for the spectral analysis of the instantaneous heart rate data. A large heart rate variability indicated by a large amplitude of the periodic biofeedback function indicates an effective vagus nerve stimulation.

In an embodiment, in the fitting, the data processing system is configured to determine the cumulative difference E between the instantaneous heart rate data and the periodic biofeedback function within the time window comprising the timepoint, and perform the fitting based on a calculated minimum of the cumulative difference E within the time window to determine the parameters of the periodic biofeedback function at the timepoint. Fitting a periodic function (here, the periodic biofeedback function) to a measurement data (here, to instantaneous heart rate data) based on a minimum of a cumulative difference is an effective way to achieve the fitting.

In an embodiment, the data processing system is configured perform the fitting within the time window with a least squares method, or a modified least squares method, or a random search, or an exhaustive search, or any combination thereof. These above mentioned “fitting methods” are effective methods for the fitting and they may be used in combination to avoid the convergence of the fitting method to a local minimum of the cumulative difference instead of a global minimum.

In an embodiment, in fitting, in determining the parameter indicating the amplitude A of the periodic biofeedback function at the timepoint, the data processing system is arranged to determine a minimum instantaneous heart rate and a maximum instantaneous heart rate of instantaneous heart rate data within the time window, subtract the minimum instantaneous heart rate from the maximum instantaneous heart rate, divide the value of subtraction by 2, and determine the parameter indicating the amplitude A of the periodic biofeedback function at the timepoint t based on the value of the division. For a periodic, alternating function such as the periodic biofeedback function this is an effective way to determine an amplitude parameter A of the function, or an initial guess for the amplitude parameter A of the function in a fitting method.

The amplitude parameter A is arranged to represent the amplitude A of the periodic biofeedback function at the timepoint and arranged determine the heart rate variability of the person at the timepoint.

The amplitude A of the instantaneous heart rate is arranged to determine the heart rate variability in frequency domain (1/time unit).

In an embodiment, in fitting, in determining the parameter indicating the angular frequency P of the periodic biofeedback function at the timepoint, the data processing system is arranged to determine a cycle time of the instantaneous heart rate data within the time window and determine the parameter indicating the angular frequency P of the periodic biofeedback function at the timepoint based on the cycle time TD. For a periodic, alternating function this is an effective way to determine an angular frequency parameter P of the function, or an initial guess for the angular frequency parameter P of the function in a fitting method.

In the present invention, the amplitude A of the instantaneous heart rate is configured to indicate or determine the heart rate variability of the person in frequency domain (1/time unit) and is thus a frequency domain characterisation. Further, also the angular frequency P is also configured to indicate or determine angular frequency P of the periodic biofeedback function at the timepoint in frequency domain.

The angular frequency P is arranged to determine the angular frequency P of the periodic biofeedback function at the timepoint in frequency domain.

Utilizing the frequency domain in the biofeedback function enables the simple and fast calculation of the parameters and further quick fitting of the periodic biofeedback function into the instantaneous heart rate data. This further enables nearly real-time feedback to the person during the monitoring session.

In an embodiment, the parameters of the periodic biofeedback function comprise a mean M of the periodic biofeedback function, and in fitting, in determining the mean M of the periodic biofeedback function at the timepoint, the data processing system is arranged to determine a mean value of the instantaneous heart rate data within the time window and determine the mean M of the periodic biofeedback function at the timepoint based on the mean value. For a periodic, alternating function such as the periodic biofeedback function this is an effective way to determine the parameter indicating the mean M of the function, or an initial guess for the parameter indicating the mean M of the function in a fitting method.

In an embodiment, the periodic biofeedback function f(t) at the timepoint comprises a sinusoidal function sin( ) such that f(t) is defined as f(t)=A sin(Pt-T)+M, in which A is the amplitude A of the instantaneous heart rate data within the time window, P is the angular frequency P of the instantaneous heart rate data within the time window, T is a time displacement, and M is a mean value M of the instantaneous heart rate data. A sinusoidal function may represent the variation of the instantaneous heart rate data well and is readily computed with digital means.

In an embodiment, the periodic biofeedback function f(t) at the timepoint comprises a skewed sinusoidal function sksin( ) such that f(t) is defined as f(t)=A sksin(Pt−T)+M=A sin [(Pt−T)+k*sin(Pt−T)], in which A is the amplitude A of the instantaneous heart rate data within the time window, P is the angular frequency P of the instantaneous heart rate data within the time window, T is a time displacement, k is a skew factor, * is a multiplication operator, and M is a mean value M of the instantaneous heart rate data. A skewed sinusoidal function is also able to account for the asymmetry of the inhale-exhale cycle of breathing and related heart rate variation as inhalation is often shorter than exhalation.

In an embodiment, the data processing system is configured to analyse the heart rate measurement data for each heartbeat measured with the heart rate sensor. This provides the most accurate set of data into which the periodic biofeedback function may be fitted.

In an embodiment, the system comprises a breathing pacer, and the breathing pacer is configured to provide feedback to the person during the health monitoring session based on the parameters of the periodic biofeedback function, on a measured breathing frequency determined from the angular frequency P, or the cumulative difference E, or any combination thereof. A breathing pacer is an advantageous unit for showing the measured breathing frequency or how well the fitting of the periodic biofeedback function to the instantaneous heart rate data is achieved. A good fitting with a high amplitude (and high heart rate variability) and small cumulative difference is indicative of a good vagus nerve stimulation.

In an embodiment, the data processing system is configured to determine a resonance score from the amplitude A of the periodic biofeedback function and from the cumulative difference E, and the breathing pacer is configured to provide feedback to the person during the health monitoring session based on the resonance score. This is an advantageous way in delivering the feedback to the person whose health is being monitored as the resonance score combines information in determining a strong vagus nerve stimulation, because it indicates the magnitude of the heart rate variability based on the amplitude A, and also the error or difference E in the determination of the amplitude A.

In an embodiment, the breathing pacer is configured to provide feedback to the person during the health monitoring session visually, or audially, or haptically, or by any combination thereof. These are advantageous ways in delivering the feedback to the person whose health is being monitored by the system.

In an embodiment, the breathing pacer comprises third software means executable on a mobile computing device, and the third software means are functionally connectable with the data processing system, and the third software means comprises computer-executable instructions for providing feedback based on the parameters of the periodic biofeedback function, or on the measured breathing frequency determined from the angular frequency P, or on the cumulative difference E, or any combination thereof. A mobile computing device like a smartphone or a tablet computer is an advantageous unit for implementing the functionality of the breathing pacer through software means.

In an embodiment, the data processing system comprises first software means executable on a mobile computing device, the first software means being functionally connectable with the heart rate sensor, and the first software means comprises computer-executable instructions for performing receiving heart rate measurement data and analysing heart rate measurement data. A mobile computing device like a smartphone or a tablet computer is an advantageous unit for implementing the functionality of data processing system through software means.

In an embodiment, the first software means comprises computer-executable instructions for storing the parameters of the periodic biofeedback function, or the first software means comprises computer-executable instructions for storing the measured breathing frequency determined from the angular frequency P, or the first software means comprises computer-executable instructions for storing the cumulative difference E, or any combination thereof. A mobile computing device like a smartphone or a tablet computer is an advantageous unit for storing the computation results of the health monitoring session, for example for a long-term analysis of the health of the person.

In an embodiment, the data processing system comprises second software means executable on a network data server, the first software means and the second software means are configured to exchange data over a network connection, and the second software means comprise computer-executable instructions for performing at least one of the steps of receiving heart rate measurement data and analyse heart rate measurement data. A data server implemented or configured, for example, in a cloud computing network or a server cluster is also an advantageous unit for analysing the heart rate measurements with a suitably fast network connection, for example a WLAN or a 3GPP standard based connection.

In an embodiment, the second software means comprises computer-executable instructions for storing the parameters of the periodic biofeedback function, or the second software means comprises computer-executable instructions for storing a measured breathing frequency that is determined from the angular frequency, or the second software means comprises computer-executable instructions for storing the cumulative difference E, or any combination thereof. The network data server is another advantageous unit for storing the computation results of the health monitoring session, for example for a long-term analysis or monitoring of the health of the person.

In an embodiment, the second software means comprises computer-executable instructions for displaying information based on the parameters of the periodic biofeedback function, or the second software means comprises computer-executable instructions for displaying information based on the cumulative difference, or both. A network data server can also provide output functionality of the analysis results, for example through a personal webpage or an applet of a mobile computing unit like smartphone.

The present invention is further based on an idea of providing a method for monitoring health of a person during a health monitoring session. The method comprises:
measuring heart beats of the person for providing heart rate measurement data with a heart rate sensor,
receiving the heart rate measurement data from the heart rate sensor into a data processing system,
analysing the heart rate measurement data in the data processing system by
   calculating instantaneous heart rate data comprising heart rate of each measured heartbeat, calculating performed over a time window in the health monitoring session, the time window comprising a timepoint, the heart rate data being calculated based on the heart rate measurement data received from the heart rate sensor,
   providing a periodic biofeedback function comprising a cumulative difference E relative to the instantaneous heart rate data and parameters, the parameters comprising:
      an amplitude A, the amplitude A determining a heart rate variability of the person at the timepoint, and
      an angular frequency P of the periodic biofeedback function at the timepoint,
   fitting the periodic biofeedback function into the instantaneous heart rate data within the time window to determine the parameters of the periodic biofeedback function at the timepoint, and the cumulative difference E at the timepoint.

Advantage of the method is that the heart rate variability of the person is determined quickly when compared to prior art methods without long datasets needed by the Fourier transform and spectral analysis.

In an embodiment, in the fitting, the method comprises determining the cumulative difference E between the instantaneous heart rate data and the periodic biofeedback function within the time window comprising the timepoint, and performing the fitting based on a calculated minimum of the cumulative difference E within the time window to determine the parameters of the periodic biofeedback function at the timepoint. Fitting a periodic function (here, the periodic biofeedback function) to a measurement data (here, to instantaneous heart rate data) based on a minimum of a cumulative difference is an effective way to achieve the fitting.

In an embodiment, the fitting is performed with a least squares method, or a modified least squares method, or a random search, or an exhaustive search, or any combination thereof. These fitting methods are effective methods for the fitting and they may be used in combination to avoid the convergence of the fitting method to a local minimum of the cumulative difference instead of a global minimum.

In an embodiment, the periodic biofeedback function f(t) at the timepoint comprises a sinusoidal function sin( ) such that f(t) is defined as f(t)=A sin(Pt−T)+M, in which A is the amplitude A of the instantaneous heart rate data within the time window, P is the angular frequency P of the instantaneous heart rate data within the time window, T is a time displacement, and M is a mean value M of the instantaneous heart rate data within the time window. A sinusoidal function may represent the variation of the instantaneous heart rate data well and is readily computed with digital means.

In an embodiment, the periodic biofeedback function f(t) at the timepoint comprises a skewed sinusoidal function sksin( ) such that f(t) is defined as f(t)=A sksin(Pt−T)+M=A sin [(Pt−T)+k*sin(Pt-T)], in which A is the amplitude A of the instantaneous heart rate data within the time window, P is the angular frequency P of the instantaneous heart rate data within the time window, T is a time displacement, k is a skew factor, * is a multiplication operator, and M is a mean value M of the instantaneous heart rate data within the time window w. A skewed sinusoidal function is also able to take into account the asymmetry of the inhale-exhale cycle of breathing and related heart rate variation as inhalation is often shorter than exhalation.

In an embodiment, the method comprises providing feedback with a breathing pacer during the health monitoring session, the feedback based on the parameters of the periodic biofeedback function, or a measured breathing frequency determined from the angular frequency P, or the cumulative difference E, or any combination thereof. Feedback helps the person or user to adjust the breathing for optimal and strong vagus nerve stimulation. A good fitting with a high amplitude and small cumulative difference is indicative of good vagus nerve stimulation.

In an embodiment, the method comprises providing feedback visually, or audially, or haptically, or any combination thereof. These are advantageous ways in delivering the feedback to the person whose health is being monitored.

In an embodiment, the method is executed in a system according to the system as defined above. The system defined above is advantageous for executing and performing the method.

As an aspect of the invention, a computer program is disclosed. The computer program comprises executable instructions which are configured to execute all the steps of a method according to the method as defined above in a computer, or a mobile computing device, or network data server, or any combination thereof. A computer program is an advantageous way to implement the method defined above.

The invention is based on the idea of determining the heart rate variability of a person from the instantaneous heart rate data with a periodic biofeedback function. A high heart rate variability indicates strong vagus nerve stimulation that may be achieved by a proper breathing rate. Best vagus nerve stimulation through breathing is achieved when the measured heart rate variation becomes periodic, has a steady and high amplitude and alternates around a mean. By fitting a periodic biofeedback function, for example a time dependent sinusoidal function with an amplitude, angular frequency, phase (time displacement) and offset (mean) value, to the instantaneous heart rate data, the heart rate variability becomes available through the amplitude of the periodic biofeedback function. In the optimal breathing frequency that maximises the vagus nerve stimulation by breathing, the amplitude of the variation of the periodic biofeedback function is maximal, and the error of cumulative difference between the fitted periodic biofeedback function and the instantaneous heart rate data is minimal. High amplitude A of the periodic biofeedback function implies high heat rate variability. Thus, high amplitude A is often alone indicative of a high heart rate variability and thus of strong vagus nerve stimulation.

In the optimal breathing frequency or in the vicinity of the optimal breathing frequency, the angular frequency P also indicates the measured breathing frequency with which the person actually breathes, f_{B}, through relation f_{B}=P/2Π.

The invention has many advantages. Determination of the heart rate variability and the optimal breathing rate for a strong vagus nerve stimulus can be done without any invasive procedures or electrical stimulation. Breathing measurements in, from or by the respiratory tract are also not needed. Instead, mere heart rate measurements of individual heartbeats are sufficient. Heart rate measurements are very convenient with modern technologies. With the fitting of the periodic biofeedback function on instantaneous heart rate data, a very quick determination of the relevant parameters of the function is also achieved as the data does not have to be subjected to complex spectral analyses like Fourier transforms that may require very long samples of heartbeat rate related information to become reliable.

With the invention, information on the heart rate variation and consequently the indication on how well various breathing rates suit the vagus nerve stimulation may become available between five to ten seconds. This is considerably faster than with prior art systems or methods.

It is to be noted that the presented invention does not disclose a diagnostic or therapeutic invention. To be a diagnostic invention, there would need to be one or more “normal” ranges of heart rate variabilities, and then one or more “abnormal” ranges of heart rate variabilities, and, then according to the invention, a determination and diagnostics should be reached based on the measured heart rate variability, and the normal and abnormal ranges. However, the present invention is not aimed into such determination.

Similarly, to be a therapeutic invention, the invention (system or method) should directly have a therapeutic effect. However, it is the breathing of the person that may stimulate the vagus nerve to a varying degree, and the strength of the stimulus may be dependent on the breathing rate. However, the invention lets the user to monitor the effect of the breathing to the heart rate variability. High heart rate variability may have health benefits and it may be indicative of improved vagus nerve stimulation, achieved through “correct” breathing. Thus, the invention allows monitoring of the person, and it is up to the user to perform the breathing through the results of the monitoring. The breathing with a certain breathing rate may then have health related positive effects.

For the purposes of this text, “vagus nerve stimulation” means “vagus nerve stimulation through breathing” that may be guided and observed by the system, method and computer program, as the system, method or computer program disclosed in the present text do not, in any way, directly stimulate the body of the person.

### BRIEF DESCRIPTION OF THE DRAWINGS

The invention is described in detail by means of specific embodiments with reference to the enclosed drawings, in which

FIG. 1 shows schematically an embodiment of the system and the person being monitored,

FIG. 2 shows concepts related to an embodiment of a periodic biofeedback function,

FIG. 3 shows schematically another embodiment of the system and the person being monitored,

FIG. 4 shows details of a fitting process between the periodic biofeedback function and the instantaneous heart rate data,

FIG. 5 shows concepts related to an embodiment of a periodic biofeedback function, the sinusoidal function,

FIG. 6 shows concepts related to an embodiment of a periodic biofeedback function, a skewed sinusoidal function,

FIG. 7 shows schematically concepts related to an embodiment of the system related to a so-called breathing pacer,

FIG. 8 shows schematically concepts related to another embodiment of the system,

FIG. 9 shows schematically concepts related to yet another embodiment of the system,

FIG. 10 shows schematically an embodiment of the method according to the current invention,

FIG. 11 shows schematically another embodiment of the method according to the current invention, and

FIG. 12 shows schematically aspects of a computer program according to an aspect of the current invention.

### DETAILED DESCRIPTION OF THE INVENTION

In the Figures and in this text, like numbers (for example 112) and like labels (for example 112m) relate to like elements.

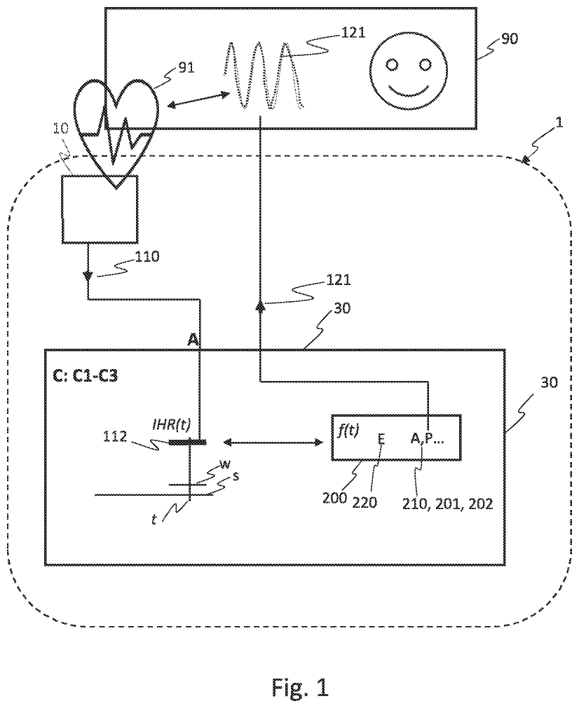

FIG. 1 shows schematically an aspect of the present invention, a system 1 for monitoring health of a person during a health monitoring session s. As discussed above, stimulating vagus nerve of a person has health effects to the person. When vagus nerve is stimulated through a relaxed, generally slow, breathing, it is important to understand how well a certain breathing rate stimulates the vagus nerve. Strong vagus nerve stimulation is often manifested with a high heart rate variability. Thus, indicating and finding a high heart rate variability (HRV) is often an indication of a strong vagus nerve stimulation. Thus, detecting a high HRV quickly and comfortably through measurements is a relevant technical problem in the fitness and health community.

For the purposes of this text, a “health monitoring session” s is any period of time during which the person is interested in observing his/her physiological matters related for example to breathing, cardiac activity or state of the nervous system, and in particular the interest of the person may be in the optimal vagus nerve stimulation.

For the purposes of this text, the terms “breathing rate” and “breathing frequency” are used interchangeably, and they all relate to a time-dependent breathing frequency of a person, calculated for example from the inverse of timespan of start of two subsequent inhalations when a person breathes. Usually breathing frequency is expressed in breathes per one minute (60 seconds), and it is usually 3-8 breaths/minute for an adult person in rest, especially when the person attempts strong or optimal vagus nerve stimulation through breathing.

For the purposes of this text, “within the time window w” means a period of time that starts and ends in the time window w, but does not necessarily span the entire time window w.

For the purposes of this text, the “instantaneous heart rate” f₁ may be defined as the inverse of the interval (in time) of two consecutive heartbeats T_{R-R}, and usually this heart rate is expressed as a frequency in minutes. The interval may be measured, for example, as the interval between timepoints of two consecutive R waves in the so-called QRS complex of a beating human heart. Thus, stated exactly, f₁=60/T_{R-R}, implying that an instantaneous heart rate of 60 beats in one minute (“BPM”, beats per minute) means that the interval between two consecutive R waves of a beating heart is 1s. Instantaneous heart rate varies from one heartbeat to the next, and this variation is called, in general, heart rate variability or heart rate variation.

Instantaneous heart rate usually varies periodically and around a mean. For example, mean of an instantaneous heart rate mean may be 70 beats in one minute, and the instantaneous heart rate may vary with an amplitude of 8 beats/minute around the mean such that minimum instantaneous heart rate is 62 beats/minute and maximum is 78 beats/minute over a period of time when the person is at rest. When the person is at rest, especially breathing, as is discussed in the present text, has the ability to alter the instantaneous heart rate.

The system 1 comprises a heart rate sensor 10 which is arranged to measure heart beats of the person 90 for providing heart rate measurement data 110. Heart of the person is indicated with symbol 91, and one of the results of the monitoring, heart rate variability (HRV) 121 is associated with symbol of periodic oscillation.

Several kinds of heart rate sensors exist, for example electrocardiogram sensors that measure the electrical activity of heart and comprise usually an elastic band or belt worn around the chest area of the person 90, and photoplethysmographic sensors that detect the heart rate based on variations of optical properties of tissue and which comprise for example a clip with a sensor worn in an earlobe or a finger of the person 90.

The system comprises also a data processing system 30 which is configured (as operation A) to receive the heart rate measurement data 110 from the heart rate sensor 10, for example during the health monitoring session s. This data may be transmitted for example in digital or analogue voltage signals, for example with electrical connections from the heart rate sensor 10 to the data processing system 30. The data processing system 30 may be any digital computer system, for example a smartphone, tablet computer, laptop computer or a digital computer.

The data processing system 30 is also (as operation C) configured to analyse the heart rate measurement data 110.

In the analysis (operation C), the data processing system 30 is configured to (as operation C1) calculate instantaneous heart rate data 112 comprising the instantaneous heart rate of each measured heartbeat. The instantaneous heart rate f₁ 112 may be defined as the inverse of the time interval of two consecutive heartbeats, as defined already above. The instantaneous heart rate data 112 is calculated based on the heart rate measurement data 110 received from the heart rate sensor 10. This is done over a time window w in the health monitoring session s. The time window w comprises a timepoint t. As an example, time window w may start at timepoint 1500s (measured from an arbitrary point in time) and end at timepoint 1540s, (making the time window 40s wide) and timepoint t can be the midmost timepoint at 1520s. In a practical implementation, the time window w may comprise discrete timepoints, for example 10, 20 or 40 discrete timepoints where the instantaneous heart rate data 112 is represented for example by digital computing device. The heath monitoring session s may comprise many, possibly overlapping time windows w. Time windows w may follow one another in time.

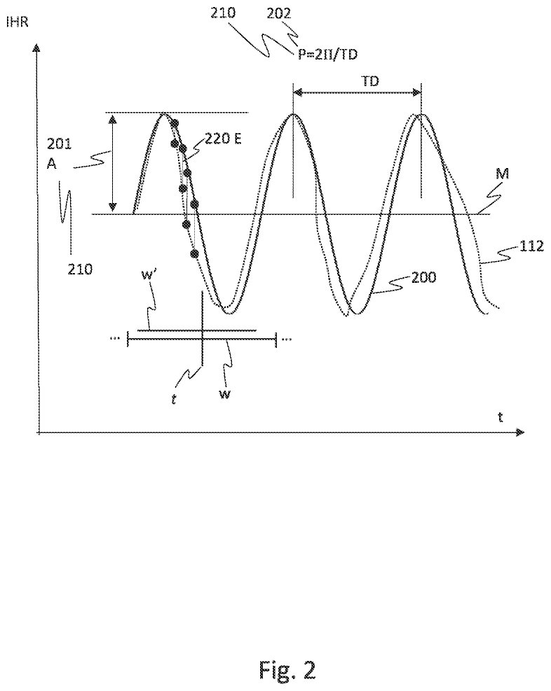

Next, referring to FIG. 2, in the analysis, the data processing system 30 (as operation C2) is configured to provide a periodic biofeedback function 200, which comprises parameters 210. The parameters 210 comprise an amplitude A 201, and an angular frequency P 202. In FIG. 2, the periodic biofeedback function is shown with solid line 200, and the instantaneous heart rate data 112 as the dotted line over a time axis t. The periodic biofeedback 200 function comprises also a cumulative difference E 220 relative to the instantaneous heart rate data 112. In FIG. 2, the cumulative difference E 220 may be the difference between the curves 200 and 112 in an arbitrary time interval w′ within the time window w. The arbitrary time interval w′ may be within the time window w completely or partially. As FIG. 2 shows, instantaneous heart rate data 112 (IHR) varies around a mean M. Maximum value of the instantaneous heart rate data 112 may be, for example, 75 beats in one minute (BPM), and minimum 63 BPM. In this case, mean value of the instantaneous heart rate is 69 BPM (average of minimum and maximum) and its amplitude of the variation is 6 BPM (maximum less minimum, divided by 2).

In the analysis, the data processing system 30 (as operation C3) is also configured to fit the periodic biofeedback function 200 into the instantaneous heart rate data 112 within the time window w to determine the parameters 210 of the periodic biofeedback function 200 at the timepoint t, and the cumulative difference E 220 at the timepoint t. The amplitude parameter A 201 indicates or determines a heart rate variability 121 of the person 90 at the timepoint t. Thus, with the system 1, determining the heart rate variability of a person 90 is achieved with no long, complex and inconvenient measurements, and quickly, because long sample periods needed by a Fourier transform are not needed.

By varying his/her the breathing rate, the person 90 may attempt to reach a high heart rate variability 121, indicative of strong vagus nerve stimulation. Thus, the health of the person 90 may be monitored through an important health indicator, the heart rate variability 121.

The data processing system 30 may be arranged to repeat the operations mentioned above for the duration of the health monitoring session s, for a plurality of time windows w. Thus, the health monitoring session s may comprise a plurality of time windows w, each comprising a timepoint t. In other words, operations A and C (C1-C3) may be repeated for the duration of the health monitoring session s for a plurality of time windows w.

In an embodiment, in the fitting (the operation C3), the data processing system 30 is configured to determine the cumulative difference E, indicated with symbol 220 between the instantaneous heart rate data 112 and the periodic biofeedback function 200 within the time window w comprising the timepoint t. The data processing system 30 is also configured perform the fitting based on a calculated minimum of the cumulative difference E 220 within the time window w to determine the parameters 210 of the periodic biofeedback function 200 at the timepoint t.

After the fitting (the operation C3), the data processing system 30 may also be configured determine the cumulative difference E 220 at the timepoint t from the calculated minimum of the cumulative difference E 220 within the time window w.

After the fitting (the operation C3), the data processing system 30 may also be configured to set the value of the cumulative difference E 220 at the timepoint t to the value of the calculated minimum of the cumulative difference E 220 within the time window w. In other words, after the fitting, the cumulative difference E 220 may have a minimum value within the time window w.

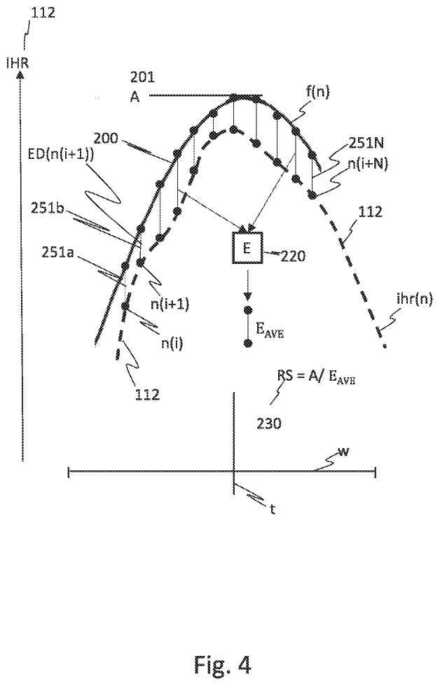

The concept of the cumulative difference E is illustrated further in FIG. 4, where instantaneous heart rate data 112 is show as dashed line curve 112. Instantaneous heart rate data 112 comprises discrete instantaneous heart rate values at discrete points in time (three discrete timepoints labelled, n(i), n(i+1), and n(i+N)) within the time window w, the time window w comprising the timepoint t. For each discrete point in time, a discrete difference ED (three discrete differences shown, marked with 251a, 251b, 251N) is determined that contributes to the cumulative difference E 220. Each of the discrete differences (251a, 251b, 251N) may be determined as the subtraction of the values of the instantaneous heart rate data 112 (represented by function ihr(n)) at the corresponding discrete timepoints n(i) . . . n(i+N), and the values of the periodic biofeedback function 200 f(n) in each of the discrete timepoints n(i) . . . n(i+N). In other words, ED(n)=ihr(n)−f(n).

In an embodiment, the data processing system 30 may be arranged to calculate the cumulative difference E 220 as the sum of absolute values of the discrete differences (251a-251N) between values of the instantaneous heart rate data 112 and values of the periodic biofeedback function 200 at the discrete timepoints n(i) . . . n(i+N),

$$E ( t ) = {\sum }_{j = i}^{N + i} \vert ihr ( n ( j ) ) - f ( n ( j ) ) \vert$$

where the | | denotes the absolute value.

As another embodiment, the data processing system 30 may be arranged to calculate the cumulative difference E 220 as the sum of squared values of the discrete differences (251a-251N) between values of the instantaneous heart rate data 112 and values of the periodic biofeedback function 200 at the discrete timepoints n,

$$E ( t ) = {\sum }_{j = i}^{N + i} {( ihr ( n ( j ) ) - f ( n ( j ) ) )}^{2} .$$

In fitting, in operation C3, the data processing system 30 may be arranged to vary the parameters 210 of the periodic biofeedback function 200, and for each set of parameters 210, determine the cumulative difference E 220. In fitting, in operation C3, the data processing system 30 may be also arranged determine the calculated minimum of the cumulative difference E 220, and the parameters 210 of periodic biofeedback function 200 used to reach the calculated minimum of the cumulative difference 220. To determine the calculated minimum of the cumulative difference E 220, the data processing system 30 is arranged to vary of the parameters 210 of the periodic biofeedback function 200 for a number of iteration loops.

The data processing system 30 may be arranged to base the variation of the parameters to one or more search algorithms, called “fitting methods” in the present text. The data processing system 30 is then configured to determine the parameters 210 resulting in a calculated minimum of the cumulative difference 220, and the parameters 210 resulting in a calculated minimum of the cumulative difference 220 are then the result of the fitting at the timepoint t, providing the values of the parameters 210 of the periodic biofeedback function 200 that give the best match between the periodic biofeedback function 200 and the instantaneous heart rate data 112 within a time window w comprising the timepoint t.

In an embodiment, the data processing system 30 may be arranged to base the varying of the parameters 210 of the periodic biofeedback function 200 to determine the calculated minimum of the cumulative difference E 220 on various search algorithms or fitting methods like least squares method, a modified least squares method, a random search method or an exhaustive search method. The data processing system 30 may be also arranged to use any combination of the search algorithms or fitting methods of the least squares method, the modified least squares method, the random search method or the exhaustive search method to determine the calculated minimum of the cumulative difference E 220.

In other words, the data processing system 30 of system 1 is configured perform the fitting C3 within the time window w with a least squares method, or a modified least squares method, or a random search, or an exhaustive search or any combination of the least squares method, the modified least squares method, the random search, and the exhaustive search.

With the random search method, the data processing system 30 is arranged to vary the parameters 210 of the periodic biofeedback function 200 randomly such that the values of the parameters 210, within ranges of feasible values, are arranged to be randomly assigned during each of the iteration loops. The data processing system 30 is then arranged to choose the set of parameters 210 reaching the calculated minimum of the cumulative difference 220 as the parameters 210 of the periodic biofeedback function 200. The data processing system 30 may also be arranged to start the random assignment of parameters from an initial guess of one or more of the parameters.

With the exhaustive search method, the data processing system 30 is arranged to vary the parameters 210 of the periodic biofeedback function 200 such that every combination of parameters 210, within ranges of feasible values in a discrete set of the feasible values, are arranged to be tried during each of the iteration loops. For example, if it is determined that the maximum amplitude A 201 of the instantaneous heart rate is 20 beats/minute, all values of A, from zero to 20 beats/minute are tried with a discrete interval of for example 0.2 beats/minute. In other words, values 0, 0.2, 0.4, . . . 20 are tried when the amplitude parameter A 201 is varied. The same variation is arranged for other parameters 210. The data processing system 30 is then arranged to choose the set of parameters 210 achieving the calculated minimum of the cumulative difference 220 as the parameters 210 of the periodic biofeedback function 200. The data processing system 30 may also be arranged to start the variation of the parameters 210 from an initial guess of one or more of the parameters.

With the least squares search method, the data processing system 30 is arranged to vary the parameters 210 of the periodic biofeedback function 200 based on the numerical Gauss-Newton algorithm to find the calculated minimum of the cumulative difference E 220. By representing the parameters 210 of the periodic biofeedback function with a vector β, and discrete difference between the periodic biofeedback function 201 and the instantaneous heart rate data 112 at discrete timepoint n(j) by a “residual” rⱼ as function of the parameters β210 (number of parameters being k), calculated minimum of the cumulative difference E 220 is found when sum of squares S(β) is minimized, where

$$S ( β ) = {\sum }_{j = i}^{N + i} {{r}_{j} ( β )}^{2} .$$

With an initial guess for the parameters 210 of β°, the data processing system 30 is arranged to vary the parameters based on an iteration rule that may be for example

$${β}^{L + 1} = {β}^{L} - {( {J}_{r}^{T} {J}_{r} )}^{- 1} {J}_{r}^{τ} r ( {β}^{L} ) ,$$

where L is the Lth iteration loop, and J is the Jacobian matrix with elements,

$${( {J}_{r} )}_{jk} = \frac{\partial {r}_{j} ( {β}^{L} )}{\partial {β}_{k}} ,$$

T denotes the matrix transpose and superscript −1 the matrix inverse.

With the modified least squares search method, the data processing system 30 is arranged to vary the parameters 210 of the periodic biofeedback function 200 based on the numerical Levenberg-Marquardt algorithm to find the calculated minimum of the cumulative difference E 220. The Levenberg-Marquardt algorithm is similar to the Gauss-Newton algorithm, but with the Levenberg-Marquardt algorithm the iteration rule may be for example

$${β}^{L + 1} = {β}^{L} - {( {J}_{r}^{T} {J}_{r} - λ I )}^{- 1} {J}_{r}^{T} r ( {β}^{L} ) ,$$

where λ is a damping parameter and I is an identity matrix. The damping factor λ may be arranged by the data processing system 30 for each of the iteration loops to a value, based on the error of the iteration, determined by the difference of the results of two consecutive iteration loops, that configures the iteration to find the parameters 210 of the periodic biofeedback function 200 faster. If the error of the iteration increases, the damping factor may be increased. If the error of the iteration decreases, the damping factor may be decreased. Clearly, with λ=0, the Levenberg-Marquardt algorithm reduces to the Gauss-Newton algorithm.

FIG. 4 illustrates also a concept of the resonance score 230. Optimal vagus nerve stimulation is achieved when the behaviour of the instantaneous heart rate data 112 is periodic and alternates over time around a mean with a large amplitude A 201. As the instantaneous heart rate data 112 is arranged to be represented by the periodic biofeedback function 200 in the data processing system 30, the cumulative difference E 220 between the periodic biofeedback function 200 and the instantaneous heart rate data 112 is also advantageous to be determined. This is because the amplitude A 201 may be large when the periodic biofeedback function 200 does not follow the measured instantaneous heart rate data 112 well. Thus, as a measure of the ability of the breathing to stimulate vagus nerve, the data processing system 30 may be configured to determine a resonance score 230 accounting for a large amplitude and good fit simultaneously. The resonance score RS 230 may be defined as a ratio of the amplitude 201 of the periodic biofeedback function 200, and an average difference E_{AVE} of the cumulative difference E 220 of N discrete timepoints within the time window w, E_{AVE}=E/N. In other words, RS=A/E_{AVE}. As another alternative, the resonance score 230 may also be defined by a two-dimensional table of values for amplitude 201 and average difference E_{AVE}, or cumulative difference E 220. The resonance score value RS 230 may be then pre-recorded for each of the A and E_{AVE} or E entries in the table, for example in the data processing system 30. Generalizing, any approach for the determination of the resonance score 230 that indicates success in the vagus nerve stimulation based on a large value of amplitude A 201 and low value of cumulative difference E 220 and wise versa is possible.

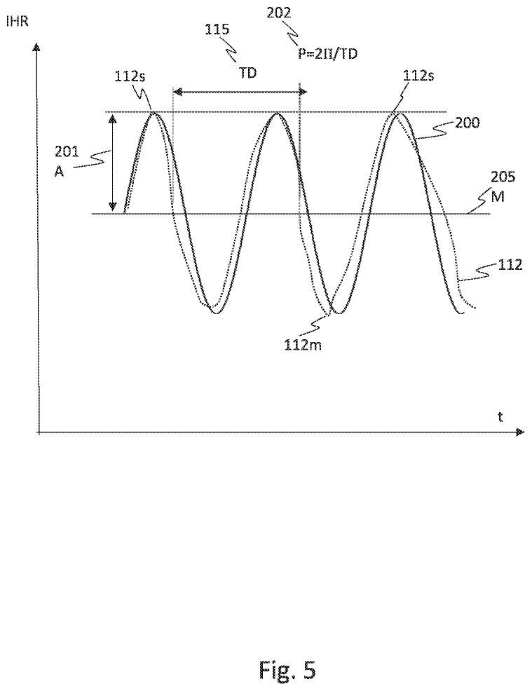

Turning to FIG. 5, in an embodiment, in fitting and in determining the parameter indicating the amplitude A 201 of the periodic biofeedback 200 function at the timepoint t, the data processing system 30 is arranged to determine a minimum instantaneous heart rate 112m and a maximum instantaneous heart rate 112s of the instantaneous heart rate data 112 within the time window w, subtract the minimum instantaneous heart rate 112m from the maximum instantaneous heart rate 112s, divide the value of subtraction by 2, and determine the parameter indicating the amplitude A 201 of the periodic biofeedback function 200 at the timepoint t based on the value of the division. The data processing system 30 may be arranged to set the amplitude parameter A 201 to value of the division directly such that for the determination of the parameters 210, in fitting, the data processing system 30 keeps parameter indicating the amplitude A 201 of the periodic biofeedback function 200 fixed. Alternatively or additionally, the data processing system 30 may use the value of the division as an initial guess in the search algorithm or fitting method.

Still referring to FIG. 5, in an embodiment, in fitting and in determining the parameter indicating the angular frequency P 202 of the periodic biofeedback 200 function at the timepoint t, the data processing system 30 is arranged to determine a cycle time TD 115 of the instantaneous heart rate data 112 within the time window w, and determine the parameter indicating the angular frequency P 202 of the periodic biofeedback function 200 at the timepoint t based on the cycle time TD 115. As shown in FIG. 5, the data processing system 30 may be arranged to determine two subsequent, essentially same values (or two values that are within a margin of error) within the instantaneous heart rate data 112 and then measure the cycle time TD between two subsequent, essentially same values, and then determine the angular frequency from a known relation P=2Π/TD. The data processing system 30 may be arranged to determine the two subsequent, essentially same values (or two values that are within a margin of error) on two successive rising or falling edges of the instantaneous heart rate data 112. The data processing system 30 may be also arranged to determine the cycle time TD between two successive maximum or two successive minimum values of the instantaneous heart rate data 112. The data processing system 30 may be arranged to set the angular frequency parameter P 202 to value based on cycle time TD directly such that for the determination of the parameters 210, in fitting, the data processing system 30 keeps parameter indicating the angular frequency P 202 of the periodic biofeedback function 200 fixed. Alternatively or additionally, the data processing system 30 may use the value of the division as an initial guess in the search algorithm or fitting method.

Still referring to FIG. 5, in an embodiment, the parameters 210 of the periodic biofeedback function 200 comprise a mean M (or mean value M) 205 of the periodic biofeedback function 200. In determining the parameter indicating the mean M 205 of the periodic biofeedback function 200 at the timepoint t, the data processing system 30 is arranged to determine a mean value of the instantaneous heart rate data 112 within the time window w and determine the parameter indicating the mean M 205 of the periodic biofeedback function 200 at the timepoint t based on the mean value. The data processing system 30 may be arranged to set the mean M 205 to the mean value directly such that for the determination of the parameters 210, in fitting, the data processing system 30 keeps the mean M 205 of the periodic biofeedback function 200 fixed. Alternatively or additionally, the data processing system 30 may use the mean value an initial guess in in the search algorithm or fitting method.

The amplitude A 201 of the instantaneous heart rate data 112 indicates or determines the heart rate variability 121 of the person 90 in frequency domain (1/time unit) and is thus a frequency domain characterisation. A measure of the heart rate variability in time domain may also be calculated as (1/(M-A))−(1/(M+A))=2A/(M²−A²)≈2A/M², where A is the amplitude A 201, and M the mean 205.

In an embodiment, and still referring to FIG. 5, the periodic biofeedback function f(t) 200 at the timepoint t comprises a sinusoidal function sin( ) such that f(t) is defined as f(t)=A sin(Pt−T)+M, in which A is the amplitude A 201 of the instantaneous heart rate data 112 within the time window w, P is the angular frequency P 202 of the instantaneous heart rate data 112 within the time window w, T is a time displacement, and M is a mean value M 205 of the instantaneous heart rate data 112. The data processing system 30 may be configured to use the sinusoidal function sin( ) and fit it to the instantaneous heart rate data 112 within the time window w to determine the parameters 210 of the periodic biofeedback function 200 at timepoint t, and subsequently the heart rate variability 121 indicated by the amplitude parameter A 201. Time displacement T is a phase parameter that is advantageous in the fitting process of a periodic biofeedback function 200 to a periodic data, the instantaneous heart rate data 112.

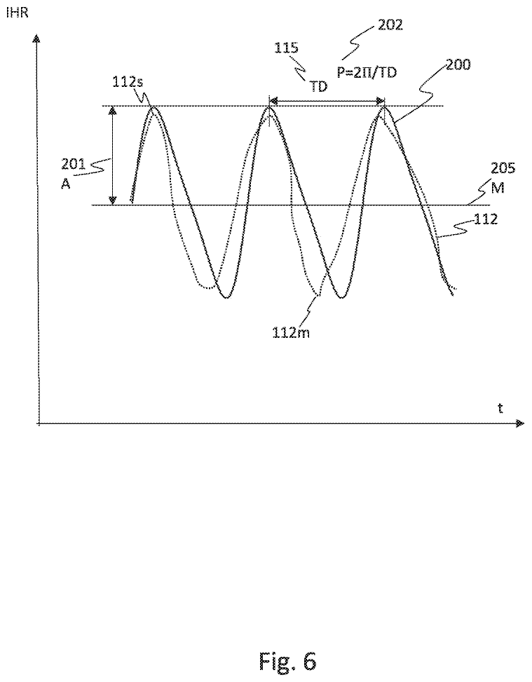

In an embodiment, and referring to FIG. 6, the periodic biofeedback function f(t) 200 at the timepoint t comprises a skewed sinusoidal function sksin( ) such that f(t) is defined as f(t)=A sksin(Pt−T)+M=A sin [(Pt−T)+k*sin(Pt−T)]+M, in which A is the amplitude A 201 of the instantaneous heart rate data 112 within the time window w, P is the angular frequency P 202 of the instantaneous heart rate data 112 within the time window w, T is a time displacement, k is a skew factor, and M is a mean value M 205 of the instantaneous heart rate data 112 within the time window w. The asterisk denotes multiplication. The skew factor k can be set to configure the periodic biofeedback function's 200 waveform to be asymmetric so that rise time (from zero value to the positive or negative amplitude value or peak value) is shorter in time than the fall time from the amplitude value back to the zero value. The skew factor may be 0-1, more advantageously 0.3-0.6 or most advantageously 0.4-0.5. A skewed sinusoidal function is advantageous as the inhalation is often somewhat shorter than the exhalation and thus the asymmetric waveform may easier be fit to the instantaneous heart rate data 112. Time displacement T is a phase parameter that is advantageous in the fitting process of a periodic biofeedback function 200 to a periodic data, the instantaneous heart rate data 112.

In an embodiment, the periodic biofeedback function f(t) 200 at the timepoint t is an alternating trapezoidal pulse train function with an amplitude A, mean value of M and angular frequency P=2Π/TD.

In an embodiment, that the data processing system 30 is configured to analyse (as operation C) the heart rate measurement data 110 for each heartbeat measured with the heart rate sensor 10.

In another embodiment, the data processing system 30 is configured to analyse, as operation C, the heart rate measurement data 110 for every second heartbeat measured with the heart rate sensor 10.

In another embodiment, the data processing system 30 is configured to analyse, as operation C, the heart rate measurement data 110 such that the frequency of analysis is based on the cumulative difference E 220 such that increasing cumulative difference E 220 makes the frequency of analysis higher, maximally every heartbeat measured with the heart rate sensor 10. Decreasing cumulative difference E 220 makes the frequency of analysis lower such that minimally, the data processing system 30 is configured to analyse, as operation C, the heart rate measurement data 110 every fifth, every tenth of every fifteenth heartbeat measured with the heart rate sensor 10.

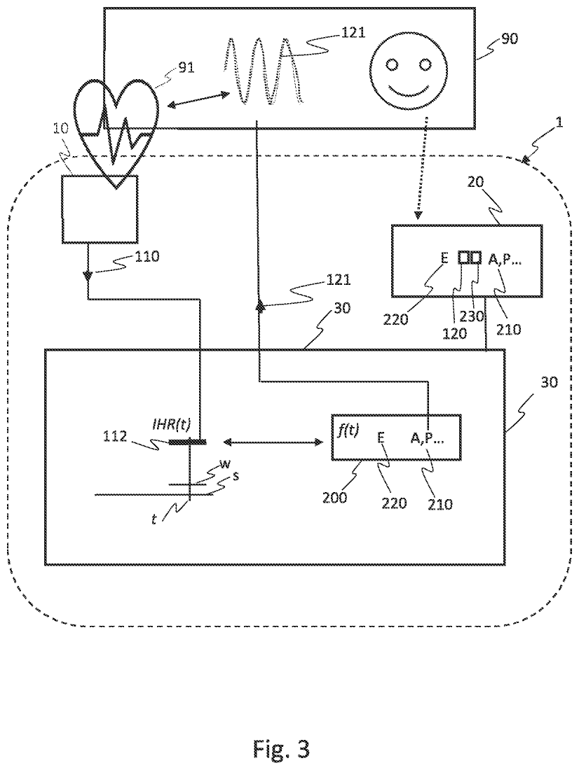

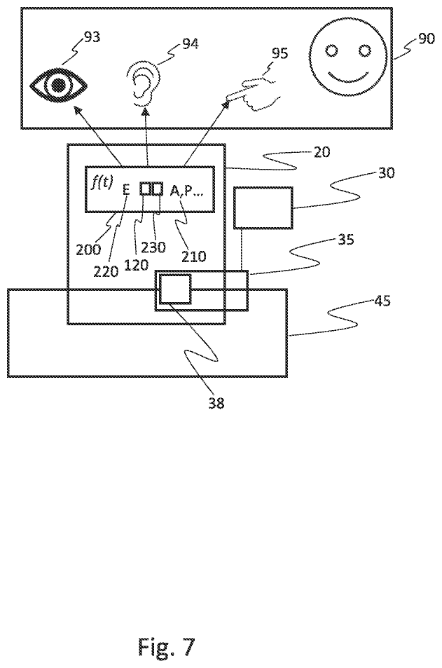

Turning to FIG. 7, in an embodiment, the system 1 comprises a breathing pacer 20. The breathing pacer 20 is configured to provide feedback to the person 90 during the health monitoring session s. The feedback may be based on the parameters 210 of the periodic biofeedback function 200. The feedback may be also based on a measured breathing frequency f_{B} 120 which is the breathing frequency of the person 90 determined from the angular frequency P 202 through relation f_{B}=P/2Π. Alternatively, the feedback may be based on the cumulative difference E 220. Still alternatively, the feedback may be based on any combination of the measured breathing frequency 120, the parameters 210 of the periodic biofeedback function 200 and cumulative difference E 220. Thus, the breathing pacer 20 may be a device, or a utility or an application or “app” arranged in for example in a mobile computing device 45 like a smartphone or a tablet computer that may indicate the measured breathing frequency 120 to the user, or the parameters 210 of the periodic biofeedback function 200 or the cumulative difference 220 as a result of the health monitoring session s or during the health monitoring session s. For example, heart rate variability 121, indicated with the amplitude A 201 of the periodic biofeedback function 200, is an indication of the person's relaxation and vagus nerve stimulation such that a “high” value of the amplitude 201 may indicate a good relaxation and strong vagus nerve stimulation, and vice versa. Relation of the breathing pacer 20 to the overall system 1 is also illustrated in FIG. 3.

The breathing pacer 20 may comprise digital electronics, analogue electronics, memory or memories, power unit like a battery, ASICs, FPGAs, digital busses, microcontrollers and microprocessors to arrange its various operations. The breathing pacer 20 may also comprise one or more input devices like a keyboard, a microphone or a touch screen display, and output devices like a LED, a display, a loudspeaker or a haptic device like a linear resonant actuator.

In an embodiment, the data processing system 30 may be configured to determine a resonance score 230 from the amplitude A 201 of the periodic biofeedback function 200 and from the cumulative difference E 220, and the breathing pacer 20 is configured to provide feedback to the person 90 during the health monitoring session s based on the resonance score 230. As presented already, the resonance score RS 230 may be defined, for example, as a ratio of the amplitude 201 of the periodic biofeedback function, and an average difference E_{AVE} of the cumulative difference E 220 of N discrete timepoints within the time window w, E_{AVE}=E/N. In other words, RS=A/E_{AVE}. As another example, the resonance score 230 may also be defined by a two-dimensional table of values for amplitude 201 and average difference E. The resonance score value RS 230 may be then pre-recorded for each of the A and E entries in the table, for example in the data processing system 30. Generalizing, any approach for the resonance score 230 that indicates success in the vagus nerve stimulation based on a large value of amplitude A 201 and low value of cumulative difference E 220 is possible.

Still referring to FIG. 7, in an embodiment, the breathing pacer 20 is configured to provide feedback or information to the person 90 during the health monitoring session s visually, indicated with symbol 93. Visual indication may comprise for example displaying the measured breathing frequency determined from the angular frequency P 202, or the determination of the moment breathing, or vagus nerve stimulation effect determined by the amplitude 201 of the periodic biofeedback function 200, or displaying the resonance score 230.

The breathing pacer 20 may be also configured to provide feedback to the person 90 during the health monitoring session s audially, indicated with symbol 94. For example, the breathing pacer 20 may be configured to play a certain tune based on the vagus nerve stimulation effect determined for example by the amplitude 201 of the periodic biofeedback function 200 or the resonance score 230.

The breathing pacer 20 may be also configured to provide feedback to the person 90 during the health monitoring session s haptically, indicated with symbol 95. For example, the breathing pacer 20 may be configured to generate vibrations periodically, for example once every hour, vibrate at a certain intensity of frequency based on the effect of vagus nerve stimulation determined for example by the amplitude 201 of the periodic biofeedback function 200, or from the resonance score 230.

The breathing pacer 20 may be also configured to provide feedback during the health monitoring session with any combination of audial, haptic or visual feedback, for example by combining the above mentioned visual, audial, and haptic feedback mechanisms.

Still referring to FIG. 7 and also illustrated in FIG. 3, in an embodiment, in the system 1, the breathing pacer 20 comprises third software means 35 executable on a mobile computing device 45, and the third software means 35 are functionally connectable with the data processing system 30. Thus, the breathing pacer 20 may be a device, or a utility arranged in for example in a mobile computing device 45 like a smartphone or a tablet computer. The third software means 35 may comprise computer-executable instructions 38 for providing feedback based on the fitting (as in operation C3) of the periodic biofeedback function 200 into the instantaneous heart rate data 112, for example feedback based on the parameters 210 of the periodic biofeedback function 200, for example the amplitude 201 of the periodic biofeedback function 200.

In an embodiment, in the system 1, the breathing pacer 20 comprises third software means 35 executable on a mobile computing device 45, and the third software means 35 are functionally connectable with the data processing system 30. Thus, the breathing pacer 20 may be a device, or a utility arranged in for example in a mobile computing device 45 like a smartphone or a tablet computer. The third software means 35 may comprise computer-executable instructions 38 for providing feedback by indicating to the person 90 the information based on the parameters 210 of the periodic biofeedback function 200.

In an embodiment, in the system 1, the breathing pacer 20 comprises third software means 35 executable on a mobile computing device 45, and the third software means 35 are functionally connectable with the data processing system 30. Thus, the breathing pacer 20 may be a device, or a utility arranged in for example in a mobile computing device 45 like a smartphone or a tablet computer. The third software means 35 may comprise computer-executable instructions 38 for providing feedback based on the parameters 210 of the periodic biofeedback function 200, the measured breathing frequency f_{B} 120 determined from the angular frequency P 202 (through relation f_{B}=P/2Π), cumulative difference E 220, or any combination thereof.

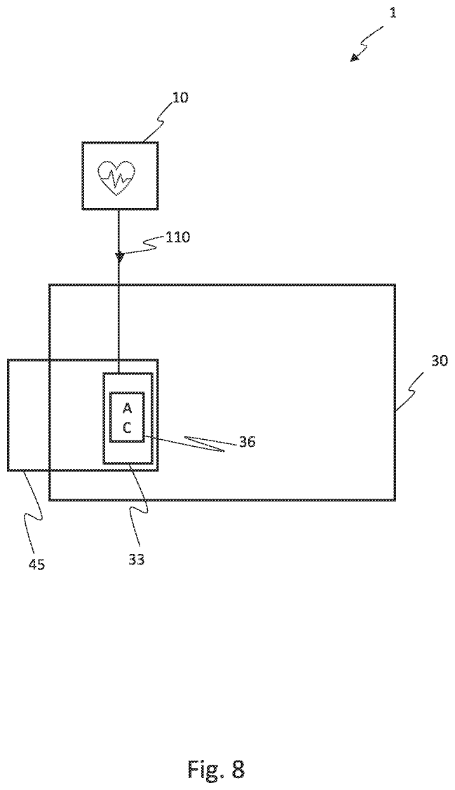

Next referring to FIG. 8, in an embodiment, in the system 1, the data processing system 30 comprises first software means 33 executable on a mobile computing device 45, the first software means 33 being functionally connectable with the heart rate sensor 10, and the first software means 33 comprises computer-executable instructions 36 for performing receiving, as operation A, heart rate measurement data 110 and analysing, as operation C, heart rate measurement data 110.

Still referring to FIG. 8, in an embodiment, in the system 1, the first software means 33 comprises computer-executable instructions 36 for storing the parameters 210 of the periodic biofeedback function 200.

In an embodiment, in the system 1, the first software means 33 comprises computer-executable instructions 36 for storing the measured breathing frequency 120 that may be derived from the angular frequency P 202.

In an embodiment, in the system 1 the first software means 33 comprises computer-executable instructions 36 for storing the cumulative difference E 220.

In an embodiment, in the system 1, the first software means 33 comprises computer-executable instructions 36 for storing any combination of the parameters 210, the measured breathing frequency 120 or the cumulative difference E 220.

Still referring to FIG. 8, in an embodiment, in the system 1, the first software means 33 comprises both computer-executable instructions 36 for storing the parameters 210 of the periodic biofeedback function 200, and computer-executable instructions 36 for storing the measured breathing frequency 120 that may be derived from the angular frequency.

As the breathing pacer 20 and the data processing system 30 may be implemented in the same unit, for example a mobile computing device 45, the breathing pacer 20 and the data processing system 30 may be functionally connected through the digital data processing units like memories, information busses, microcontrollers and microprocessors of the mobile computing device 45.

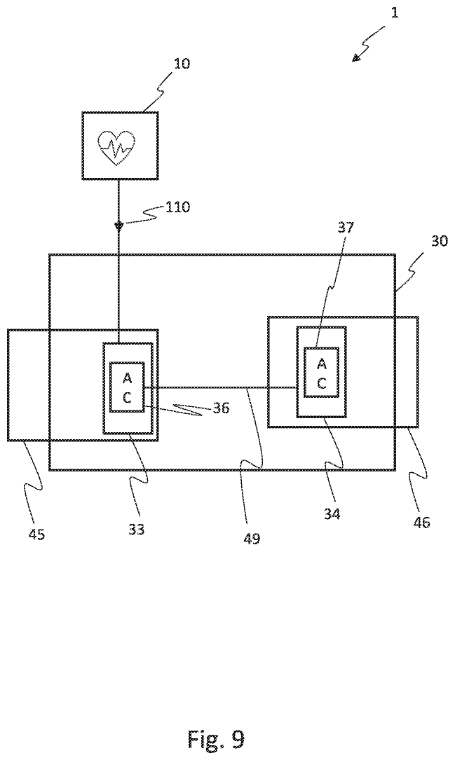

Next referring to FIG. 9, in an embodiment, in the system 1, the data processing system 30 comprises second software means 34 executable on a network data server 46, the first software means 33 and the second software means 34 are configured to exchange data over a network connection 49, and the second software means 34 comprise computer-executable instructions 37 for performing at least one of the operations of receiving, as operation A, heart rate measurement data, and analyse, as operation C, heart rate measurement data. The network connection 49 may comprise physical conductors or a radio interface or both, and it may be arranged through one or more of the standards of ethernet, wireless LAN, Bluetooth, GSM, 3GPP or other cable-based or wireless standard.

Still referring to FIG. 9, in an embodiment, in the system 1, the second software means 34 comprises computer-executable instructions 37 for storing the parameters 210 of the periodic biofeedback function 200.

In an embodiment, in the system 1, the second software means 34 comprises computer-executable instructions 37 for storing the measured breathing frequency 120 that may be derived from the angular frequency 202.

In an embodiment, in the system 1 the second software means 34 comprises computer-executable instructions 37 for storing the cumulative difference E 220.

In an embodiment, in the system 1, the second software means 34 comprises computer-executable instructions 37 for storing any combination of the parameters 210, the measured breathing frequency 120 or the cumulative difference E 220.

Still referring to FIG. 9, in an embodiment, in the system 1, the second software means 34 comprises computer-executable instructions 37 for storing the parameters 210 of the periodic biofeedback function 200, and second software means 34 also comprises computer-executable instructions 37 for storing the measured breathing frequency f_{B} 120 that may be derived from the angular frequency P through relation f_{B}=P/2Π.

In an embodiment, in the system 1, the second software means 34 comprises computer-executable instructions 37 for displaying information based on the parameters 210 of the periodic biofeedback function 200. This is advantageous for example if the health monitoring results are to be published for example over the internet or in an app of a smartphone.

In an embodiment, in the system 1, the second software means 34 comprises computer-executable instructions 37 for displaying information based on the cumulative difference E 220. This is advantageous for example if the health monitoring results are to be published for example over the internet or in an app of a smartphone.

In an embodiment, in the system 1, the second software means 34 comprises computer-executable instructions 37 for displaying information based on the parameters 210 of the periodic biofeedback function 200, and the second software means 34 comprises computer-executable instructions 37 for displaying information based on the cumulative difference E 220. This is again advantageous for example if the health monitoring results are to be published for example over the internet or in an app of a smartphone.

In an embodiment, in the system 1, the second software means 34 comprises computer-executable instructions 37 for displaying the measured breathing frequency 120 determined from the angular frequency P 202. This is also advantageous for example if the health monitoring results are to be published for example over the internet or in an app of a smartphone.

In an embodiment, in the system 1, the second software means 34 comprises computer-executable instructions 37 for displaying information based on the parameters 210 of the periodic biofeedback function 200 and the second software means 34 also comprises computer-executable instructions 37 for displaying the measured breathing frequency 120 defined from the angular frequency P 202. This is again advantageous for example if the health monitoring results are to be published for example over the internet or in an app of a smartphone.

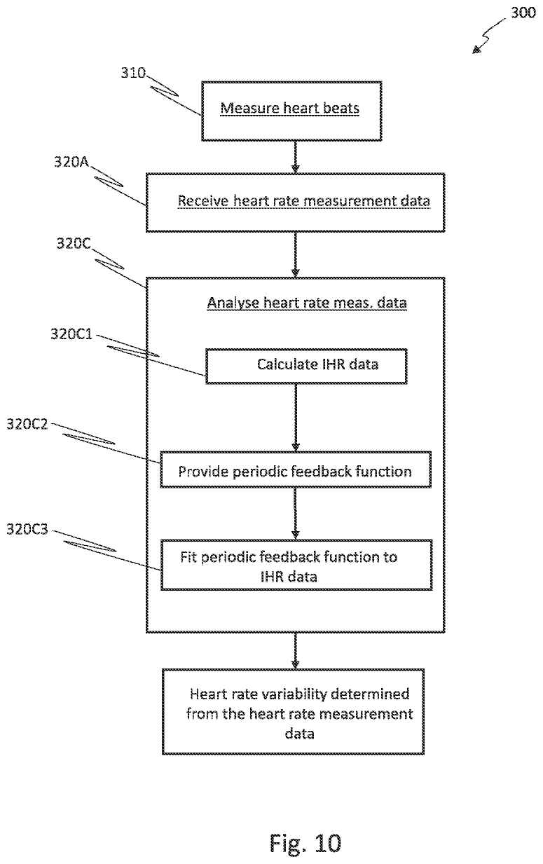

Next turning to FIG. 10 and also to FIG. 1, as an aspect of the invention, method 300 for monitoring health of a person 90 during a health monitoring session s is disclosed. The method 300 comprises: in step 310, measuring 310 heart beats of the person 90 for providing heart rate measurement data 110 with a heart rate sensor 10. The method also comprises, in step 320A, receiving the heart rate measurement data 110 from the heart rate sensor 10 into a data processing system 30. In step 320C, the method comprises analysing 320C the heart rate measurement data 110 in the data processing system 30. Analysing, step 320C, comprises, in step 320C1, calculating instantaneous heart rate data 112 comprising instantaneous heart rate of each measured heartbeat, the instantaneous heart rate data 112 calculated based on the heart rate measurement data 110 received from the heart rate sensor 10. Calculation is performed over a time window w in the health monitoring session s. The time window w comprises a timepoint t. Analysis, step 320C, comprises further providing, in step 320C2 a periodic biofeedback function 200. The periodic biofeedback function 200 comprises parameters 210, and the parameters 210 comprise an amplitude A 201, and an angular frequency P 202. The periodic biofeedback 200 function comprises also a cumulative difference E 220 relative to the instantaneous heart rate data 112. Analysis, step 320C, comprises also, as step 320C3, fitting the periodic biofeedback function 200 into the instantaneous heart rate data 112 within the time window w to determine the parameters 210 of the periodic biofeedback function 200 at the timepoint t, and the cumulative difference E 220 at the timepoint t. As an outcome of said process, the amplitude A 201 determines a heart rate variability 121 of the person 90 at the timepoint t.

The method may comprise also repeating the steps mentioned above for the duration of the health monitoring session s, for a plurality of time windows w. Thus, the health monitoring session s may comprise a plurality of time windows w, each comprising a timepoint t. In other words, steps 320A, 320C (320C1-320C3) and 310 may be repeated for the duration of the health monitoring session s for a plurality of time windows w.

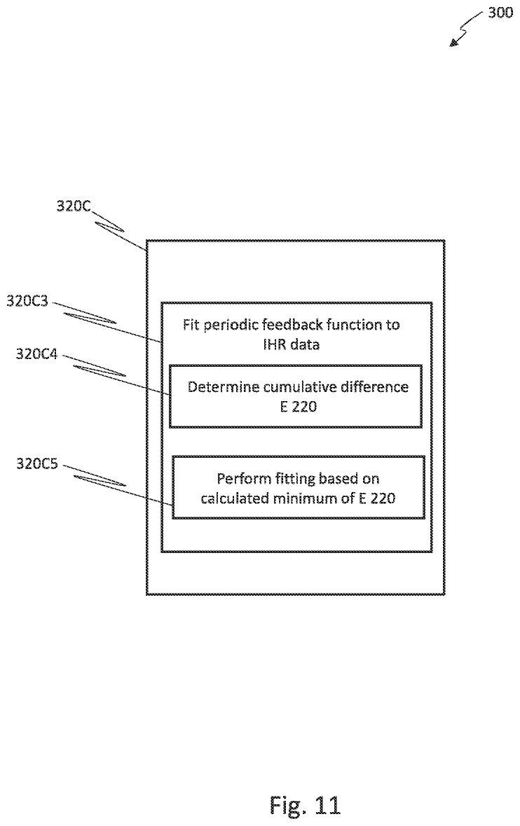

As shown in FIG. 11, as an embodiment, in the fitting, in step 320C3, the method 300 comprises determining, in step 320C4, a cumulative difference E 220 between the instantaneous heart rate data 112 and the periodic biofeedback function 200 within the time window w comprising the timepoint t, and performing, in step 320C5, the fitting based on a calculated minimum of the cumulative difference E 220 within the time window w to determine the parameters 210 of the periodic biofeedback function 200 at the timepoint t.

The method 300 may also comprise, after step 320C5, determining the cumulative difference E 220 at the timepoint t from the calculated minimum of the cumulative difference E 220 within the time window w.

The method 300 may also comprise, after step 320C5, setting the value of the cumulative difference E 220 at the timepoint t to the value of the calculated minimum of the cumulative difference E 220 within the time window w. In other words, after the fitting, the cumulative difference E 220 may have a minimum value within the time window w.

In an embodiment, in step 320C5, the method 300 comprises performing the fitting within the time window w with a least squares method, a modified least squares method, a random search, an exhaustive search, or any combination thereof. The least squares method, the modified least squares method, the random search, and the exhaustive search may be called search algorithms, search methods or fitting methods. Operation of these methods is discussed already above in relation to system 1.

In an embodiment, and referring back to FIG. 5, in the method 300, the periodic biofeedback function f(t) 200 at the timepoint t comprises a sinusoidal function sin( ) such that f(t) is defined as f(t)=A sin(Pt−T)+M, in which A is the amplitude A 201 of the heart instantaneous heart rate data 112 within the time window w, P is the angular frequency P 202 of the instantaneous heart rate data 112 within the time window w, T is a time displacement, and M is a mean value M 205 of the instantaneous heart rate data 112.

In an embodiment, and referring back to FIG. 6 in the method 300, the periodic biofeedback function f(t) 200 at the timepoint t comprises a skewed sinusoidal function sksin( ) such that f(t) is defined as f(t)=A sksin(Pt−T)+M=A sin [(Pt−T)+k*sin(Pt−T)], in which A is the amplitude A 201 of the instantaneous heart rate data 112 within the time window w, P is the angular frequency P 202 of the instantaneous heart rate data 112 within the time window w, T is a time displacement, k is a skew factor, and M is a mean value M 205 of the instantaneous heart rate data 112 within the time window w. The skew factor k can be set to configure the periodic biofeedback function waveform asymmetric so that, for example, rise time (from zero value to the positive or negative amplitude value or peak value) is shorter in time than the fall time from the amplitude value back to the zero value. This is advantageous as the inhalation is often somewhat shorter than the exhalation and thus the asymmetric waveform may easier be fit to the instantaneous heart rate data 112. The skew factor may be 0-1, more advantageously 0.3-0.6 or most advantageously 0.4-0.5.

In an embodiment, the method 300 comprises providing feedback with a breathing pacer 20 (as shown FIGS. 3 and 7) during the health monitoring session s. The feedback may be based on the parameters 210 of the periodic biofeedback function 200, on a measured breathing frequency 120 determined from the angular frequency P 202, or on the cumulative difference E 220, or on any combination of the parameters 210 of the periodic biofeedback function 200, cumulative difference E 220, and the measured breathing frequency 120.

Referring back to FIG. 7, in an embodiment, the method 300 comprises providing feedback visually 93, audially 94, haptically 95 or by any combination thereof.

The method 300 defined above may be executed in the system 1 as defined above.

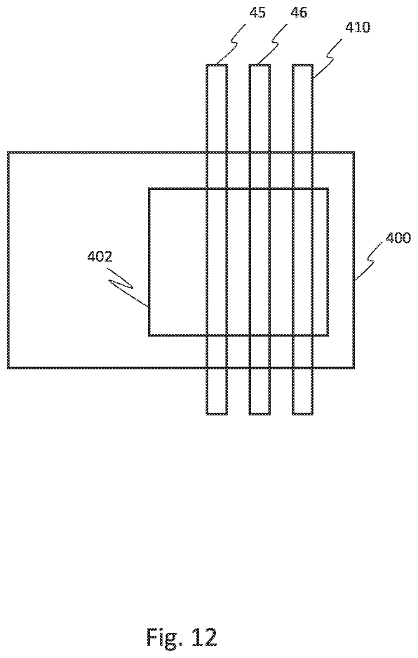

Next turning to FIG. 12, as an aspect of the present invention, a computer program 400 comprises executable instructions 402 which are configured to execute the steps of the method 300 in a computer 410, or in a mobile computing device 45 or network data server 46, or any combination thereof.

For the purposes of this text, the data processing system 30 may comprise digital electronics, analogue electronics, memory or memories, power unit like a battery, ASICs, FPGAs, digital busses, microcontrollers and microprocessors to arrange its various operations. The data processing system 30 may also comprise one or more input devices like a keyboard, a microphone or a touch screen display, and output devices like a LED, a display, a loudspeaker or a haptic device like a linear resonant actuator. The data processing system 30 may be arranged in a mobile computing device like a smartphone or tablet computer, for example through software and hardware means. If the heart rate measurement data from the heart rate sensor 10 is in analogue format, the data processing system 30 may also comprise analogue-to-digital converters to arrange conversion of an analogue signal into a digital signal for further processing. Communication between different units may be arranged with known communication interfaces like I2C, SPI, ethernet or radio interfaces like WLAN, Bluetooth or 3GPP.

The invention has been described above with reference to the examples shown in the figures. However, the invention is in no way restricted to the above examples but may vary within the scope of the claims.

1.-25. (canceled)

26. A system for monitoring health of a person during a health monitoring session (s), wherein the system comprises:
a heart rate sensor arranged to measure heart beats of the person for providing heart rate measurement data, and
a data processing system configured to:
receive (A) the heart rate measurement data from the heart rate sensor during the health monitoring session (s), and
analyse (C) the heart rate measurement data such that in the analysis, the data processing system is configured to:
calculate (C1) instantaneous heart rate data comprising heart rate of each measured heartbeat, the calculating being performed over a time window (w) in the health monitoring session (s), the time window (w) comprising a timepoint (t), the instantaneous heart rate data being calculated based on the heart rate measurement data received from the heart rate sensor,
provide (C2) a periodic biofeedback function comprising parameters and a cumulative difference (E) relative to the instantaneous heart rate data, the parameters comprising:
an amplitude (A) determining a heart rate variability of the person at the timepoint (t), and
an angular frequency (P) of the periodic biofeedback function at the timepoint (t),
fit (C3) the periodic biofeedback function into the instantaneous heart rate data within the time window (w) to determine:
the parameters of the periodic biofeedback function at the timepoint (t), and
the cumulative difference (E) at the timepoint (t).

27. The system according to claim 26, wherein during the fitting (C3), the data processing system is configured to:
determine the cumulative difference (E) between the instantaneous heart rate data and the periodic biofeedback function within the time window (w) comprising the timepoint (t), and
perform the fitting (C3) based on a calculated minimum of the cumulative difference (E) within the time window (w) to determine the parameters of the periodic biofeedback function at the timepoint (t).

28. The system according to claim 27, wherein:
the data processing system is configured to perform the fitting (C3) within the time window (w) with at least one of:
a least squares method; or
a modified least squares method; or
a random search; or
an exhaustive search; or
any combination thereof.

29. The system according to claim 26, wherein fitting (C3), in determining the parameter indicating the amplitude (A) of the periodic biofeedback function at the timepoint (t), the data processing system is arranged to:
determine a minimum instantaneous heart rate and a maximum instantaneous heart rate of instantaneous heart rate data within the time window (w),
subtract the minimum instantaneous heart rate from the maximum instantaneous heart rate,
divide the value of subtraction by 2, and
determine the parameter indicating the amplitude (A) of the periodic biofeedback function at the timepoint (t) based on the value of the division.

30. The system according to claim 27, wherein fitting (C3), in determining the parameter indicating the angular frequency (P) of the periodic biofeedback function at the timepoint (t), the data processing system is arranged to:
determine a cycle time (TD) of the instantaneous heart rate data within the time window (w), and
determine the parameter indicating the angular frequency (P) of the periodic biofeedback function at the timepoint (t) based on the cycle time (TD).

31. The system according to claim 26, wherein:
the parameters of the periodic biofeedback function comprise a mean (M) of the periodic biofeedback function, and
in fitting (C3), in determining the mean (M) of the periodic biofeedback function at the timepoint (t), the data processing system is arranged to:
determine a mean value of the instantaneous heart rate data within the time window (w), and
determine the mean (M) of the periodic biofeedback function at the timepoint (t) based on the mean value.

32. The system according to claim 26, wherein:
the periodic biofeedback function f(t) at the timepoint (t) comprises a sinusoidal function sin( ) such that f(t) is defined as f(t)=A sin(Pt−T)+M, in which:
A is the amplitude A of the instantaneous heart rate data within the time window (w),
P is the angular frequency P of the instantaneous heart rate data within the time window (w),
T is a time displacement, and
M is a mean value M of the instantaneous heart rate data; or
the periodic biofeedback function f(t) at the timepoint (t) comprises a skewed sinusoidal function sksin( ) such that f(t) is defined as f(t)=A sksin(Pt−T)+M=A sin[(Pt−T)+k*sin(Pt−T)], in which:
A is the amplitude A of the instantaneous heart rate data within the time window (w),
P is the angular frequency P of the instantaneous heart rate data within the time window (w),
T is a time displacement,
k is a skew factor, * is a multiplication operator, and
M is a mean value M of the instantaneous heart rate data.

33. The system according to claim 26, wherein the data processing system is configured to analyse (C) the heart rate measurement data for each heartbeat measured with the heart rate sensor.

34. The system according to claim 26, wherein
the system comprises a breathing pacer, and
the breathing pacer is configured to provide feedback to the person during the health monitoring session (s) is based on at least one of:
the parameters of the periodic biofeedback function; or
the cumulative difference (E); or
a measured breathing frequency determined from the angular frequency (P); or
any combination thereof.

35. The system according to claim 34, wherein:
the data processing system is configured to determine a resonance score from the amplitude A of the periodic biofeedback function and from the cumulative difference (E), and
the breathing pacer is configured to provide feedback to the person during the health monitoring session (s) based on the resonance score.

36. The system according to claim 34, wherein the breathing pacer is configured to provide feedback to the person during the health monitoring session (s) by at least one of:
visually; or
audially; or
haptically; or
any combination thereof.

37. The system according to claim 34, wherein:
the breathing pacer comprises third software means executable on a mobile computing device, and
the third software means are functionally connectable with the data processing system, and:
the third software means comprises computer-executable instructions for providing feedback based on:
the parameters of the periodic biofeedback function; or
the measured breathing frequency determined from the angular frequency (P); or
the cumulative difference (E); or
any combination thereof.

38. The system according to claim 26, wherein:
the data processing system comprises first software means executable on a mobile computing device, the first software means being functionally connectable with the heart rate sensor, and
the first software means comprises computer-executable instructions for performing receiving (A) heart rate measurement data and analysing (C) heart rate measurement data.

39. The system according to claim 38, wherein:
the first software means comprises computer-executable instructions for storing the parameters of the periodic biofeedback function; or
the first software means comprises computer-executable instructions for storing the measured breathing frequency determined from the angular frequency (P); or
the first software means comprises computer-executable instructions for storing the cumulative difference (E); or
any combination thereof.

40. The system according to claim 38, wherein:
the data processing system comprises second software means executable on a network data server,
the first software means and the second software means are configured to exchange data over a network connection, and
the second software means comprise computer-executable instructions for performing at least one of the steps of receiving (A) heart rate measurement data and analyse (C) heart rate measurement data.

41. The system according to claim 40, wherein:
the second software means comprises computer-executable instructions for storing the parameters of the periodic biofeedback function; or
the first software means comprises computer-executable instructions for storing the cumulative difference (E); or
any combination thereof.

42. The system according to claim 41, wherein:
the second software means comprises computer-executable instructions for displaying information based on the parameters of the periodic biofeedback function; or
the second software means comprises computer-executable instructions for displaying information based on the cumulative difference (E); or both.

43. A method for monitoring health of a person during a health monitoring session (s), wherein the method comprises:
measuring heart beats of the person for providing heart rate measurement data with a heart rate sensor,
receiving the heart rate measurement data from the heart rate sensor into a data processing system,
analysing the heart rate measurement data in the data processing system by:
calculating instantaneous heart rate data comprising heart rate of each measured heartbeat, calculating performed over a time window (w) in the health monitoring session (s), the time window (w) comprising a timepoint (t), the heart rate data being calculated based on the heart rate measurement data received from the heart rate sensor,
providing a periodic biofeedback function comprising a cumulative difference E) relative to the instantaneous heart rate data and parameters, the parameters comprising:
an amplitude (A) determining a heart rate variability of the person at the timepoint (t), and
an angular frequency (P) of the periodic biofeedback function at the timepoint (t), the periodic biofeedback function comprising a cumulative difference (E) relative to the instantaneous heart rate data, and
fitting the periodic biofeedback function into the instantaneous heart rate data within the time window (w) to determine the parameters of the periodic biofeedback function at the timepoint (t), and the cumulative difference (E) at the timepoint (t).

44. A processor-readable medium storing instructions which, when executed by at least one processor of an apparatus, cause the apparatus at least to perform the method according to claim 43.
